(19) United States
(12) Patent Application Publication
(10) Pub. No.: US 2025/0380946 A1
(43) Pub. Date: Dec. 18, 2025

(54) ANASTOMOSIS CLIP

(71) Applicant: AMT MEDICAL B.V., EDE (NL)

(72) Inventor: Glenn BRONKERS, EDE (NL)

(21) Appl. No.: 18/875,998

(22) PCT Filed: Jun. 20, 2023
(86) PCT No.: PCT/EP2023/066588

§ 371 Date: Dec. 17, 2024

(30) Foreign Application Priority Data
Jun. 20, 2022 (NL) 2032224

(51) Int. Cl. A61B 17/11

(52) U.S. Cl. CPC A61B 17/11; A61B 2017/1107; A61B 2017/1132; A61B 2017/1135; A61B 2017/1139

(57) ABSTRACT

The invention relates to an anastomosis clip (100; 200) for connecting a first vessel to the side wall of a second vessel. The clip comprises a two prong fork (2) with fork base (3) and two prongs (4, 5) extending therefrom to a tapered end part (6) having a sharp tip (7), a ring (1), and a clamping structure (10; 60) to clamp the fork towards the ring. The ring and the fork are movable with respect to each other from an open position to the closed position. In the closed position the prongs extend along the ring plane. The ring has, for each of said prongs an associated supporting area (21, 22). In the closed position the tapered part of the prongs is supported by its associated supporting area such that the sharp tips are shielded by the associated support area.

The invention relates to the field of anastomosis clips for connecting vessels in a human or animal body. The vessels may be blood vessels, like natural and/or artificial blood vessels. In case of natural blood vessels, these may be native vessels of the patient herself/himself or these may be donor vessels harvested from other humans or animals. Both in case of native vessels or donor vessels, the vessels optionally may have been treated.

### BACKGROUND OF THE INVENTION

Anastomoses can be sub-divided into three groups: ETS-anastomoses (ETS=end-to-side), STS-anastomoses (STS=side-to-side), and ETE-anastomoses (ETE=end-to-end).

Traditionally, an anastomosis is established by sewing two vessels together. This is laboursome. Many anastomosis tools, such as clips, have been developed to facilitate or even avoid this labour-intensive process of sewing.

Some examples of such tools and clips can be found in EP 2.265.192, EP 2.663.243, WO-2011/0642495, and WO 2013/105.848. These tools/clips have a ring structure and a two prong fork with sharp tips at the free ends of the prongs. The two prong fork is pierced from the outside through the side wall of the blood vessel into the inside of the vessel—the S-vessel of an ETS-anastomosis or STS-anastomosis—and may subsequently also be pierced (back) from the inside of the vessel through the side wall to the outside of the vessel. The ring structure serves as an aid for attaching the other one the vessels of the anastomosis to be established. The other one of the vessels may be attached to the ring structure or the ring structure may be an intermediate part lying so to say in between the first vessel of the anastomosis and the second vessel of the anastomosis and assisting in establishing the anastomosis. Already before and during establishing the anastomosis but also after having established the anastomosis, the sharp tips at the free ends of the prongs may cause damage to the vessel (to be) connected or to surrounding tissue.

### SUMMARY OF THE INVENTION

One object of the invention to provide an alternative and/or improved anastomosis clip. Another object of the invention is to provide an anastomosis clip overcoming the above problem associated to the sharp tips of the prongs.

According to the invention one or more of the above objects are achieved by providing an anastomosis clip for connecting a first vessel to the side wall of a second vessel, wherein the anastomosis clip comprises:
a first two prong fork having a fork base and two prongs extending from the fork base to a tapered end part, the tapered end parts tapering from a taper base to a sharp tip,
a ring structure defining a ring plane, and
a clamping structure configured to clamp the first fork in a closed position with a first clamping force towards and/or against the ring structure;
   wherein the ring structure and the first fork are movable with respect to each other from an open position to the closed position;
   wherein, in the open position, the ring structure and the first fork are arranged at a distance from each other;
   wherein, in the closed position:
the prongs of the first fork extend along the ring plane, and
the fork base of the first fork is located at a base side of the ring structure whilst the tapered end parts of the prongs of the first fork are located at a prong end side of the ring structure, the prong end side being, viewed along the ring plane, opposite the base side;
   wherein the ring structure has, for each of said prongs of the first fork, an associated supporting area associated to a said prong of the first fork;
   wherein each said supporting area:
has, viewed with respect to the fork base of the first fork, a distal end and a proximal end, and
is configured to support a part of the associated prong when the first fork is, in the closed position, clamped with the first clamping force towards the ring structure; and
   wherein, in the closed position, a first distance measured from the fork base of the first fork to the sharp tip of the tapered end part of a said prong is at most as long as a second distance measured from the fork base of the first fork to the distal end of the associated supporting area such that, viewed in a transverse direction transverse to the ring plane, each said sharp tip is shielded and/or supported by its associated supporting area.

Both the first vessel and the second vessel of the anastomosis may according to the invention be a blood vessel. According to the invention, both vessels may be a natural vessel, both vessels may be an artificial vessel, or one vessel may be a natural vessel whilst the ither is an artificial vessel. In case of a natural vessel, the vessel may according to the invention be a native vessel from the patient—optionally harvested from the patient somewhere else than the location of the anastomosis—or a donor vessel. Both in case of native vessels or donor vessels, the vessels optionally may have been treated.

In the first condition, the clamping structure may clamp the first fork towards and/or against the ring structure. When the anastomosis is established, vessel wall of one of the vessels of the anastomosis will in general lie between the first fork and the ring structure. Further, the clamping may according to the invention be established in any manner allowing the first fork and the ring structure to be held the first fork and ring structure in a fixed position relative to each other with vessel wall in between. The clamping may:
according to a first example of the invention for example be established by a spring effecting a spring force clamping the ring structure and first fork together, and/or
according to a second example of the invention be established by a snap action of one or more first snap members provided on one of the ring structure and first fork and one or more second snap members provided on the other one of the ring structure and the first fork, and/or
according to a third example of the invention be established by one or more clamping brackets engaging on the ring structure and first fork to keep those together.

In the closed position the ring structure and the first fork will be connected to each other to form a unit. In the open position of the ring structure and first fork:
these may be form one unit, the ring structure and the first fork for example being connected by a hinge and/or spring, or
these may be separate parts to be connected to each other during establishing the anastomosis for example by means of one or more sets of cooperating snap members and/or one or more clamping brackets.

By configuring the supporting areas and the first fork in mutual relation such that a first distance measured from the fork base of the first fork to the sharp tip of the tapered end part of a said prong is at most as long as a second distance measured from the fork base of the first fork to the distal end of the associated supporting, it is ensured that, at least after having established the anastomosis, the sharp tips are shielded by the associated supporting areas and that the tapered end part finds support on the associated supporting area because the end part of the tapered end part is, viewed transverse to the ring plane, overlapped by the associated supporting area. The shielding of the tips prevents the tips from damaging the vessels of the anastomosis and other tissue. The end parts of the tapered end parts being overlapped by the associated supporting areas prevents the tapered end parts from being deformed or bended due to the clamping force exerted by the clamping structure.

According to a further embodiment of the invention, this ‘end part of the tapered end part which is overlapped by the associated supporting area’ may have—viewed in length direction of the prong—a length of about 35% or more, such as 50% or more, of the length of the taper of the tapered end part, in other words the associated supporting area overlaps at least about 35%, such as at least about 50%, of the tapered end part. In the closed position, the supporting area may overlap with for example about 75% or more of the associated tapered end part. With respect to the tapered end part, it is to be noted that due to the taper, the cross section of the prongs—transverse to the length direction of the prongs—decreases in distal direction with the consequence that these tapered parts are more susceptible to deforming and/or bending. Such deforming/bending may result in the sharp tips pointing away from the ring structure and consequently increasing the risk of damaging a vessel or surrounding tissue.

The supporting area defines, viewed from its proximal end to its distal end, an area length. This area length may according to an embodiment of the invention have about the same length as—so to say in the range of 95-105% of—the length of the tapered end part.

According to a further additional or alternative embodiment of the invention, the supporting area(s) may project, viewed in the closed position and in the distal direction, beyond the associated sharp tip(s). This ensures the sharp tip being well shielded from vessel tissue and other surrounding tissue.

The area length may, according to a further embodiment, be at least 1.5× the thickness of the prongs of the fork. In a one example, the area length may be at least 2.0× the thickness of the prongs of the first fork. In another example, the area length may be at least 2.5× the thickness of the prongs of the first fork.

The area length may, according to a further embodiment, be at most 6× the thickness of the prongs of the first fork. For example, the area length may be at most 5× the thickness of the prongs of the first fork.

According to a further embodiment of the invention, the area length is equal to or larger than the length of the tapered end part. This ensures the tapered end part being well supported.

The area length may, according to a further embodiment, be at least 110% of the length of the tapered end part. This might go together with the area length being at most 120% or at most 115% of the length of the tapered end part.

The area length may, according to a further embodiment, be at least 115% of the length of the tapered end part. This might go together with the area length being at most 125% or at most 120% of the length of the tapered end part.

The area length may, according to a further embodiment, be at least 120% of the length of the tapered end part. This might go together with the area length being at most 130% or at most 125% of the length of the tapered end part.

The area length may, according to a further embodiment, be at least 125% of the length of the tapered end part. This might go together with the area length being at most 135% or at most 130% of the length of the tapered end part.

According to a further embodiment of the invention, a third distance from the fork base of the first fork to the taper base of the tapered end part of a said prong is, in the closed position, at least as long as a fourth distance from the fork base of the first fork to the proximal end of the associated supporting area such that, viewed in the transverse direction, each said taper base is supported by its associated supporting area. By configuring the supporting areas and the first fork in mutual relation such that a third distance measured from the fork base of the first fork to the taper base of the tapered end part of a said prong is at least as long as a fourth distance measured from the fork base of the first fork to the proximal end of the associated supporting, it is ensured that (at least) after having established the anastomosis, not only the sharp tips are shielded by the associated supporting areas but also basically the entire tapered end part is, viewed transverse to the ring plane, well and reliably overlapped by the associated supporting area. The tapered end parts being basically fully overlapped by the associated supporting areas prevents the tapered parts from being deformed or bended due to the clamping force exerted by the clamping structure. With respect to ‘being basically fully overlapped’ it is noted that each associated supporting area may be a discontinuous supporting area having for example two supporting sub-areas spaced by a gap above which the support is actually interrupted.

According to a further embodiment of the invention, the tapered end parts of the prongs of the first fork each have a first taper with a first taper direction and a second taper with a second taper direction, wherein the first taper direction is different from the second taper direction, and wherein, viewed in length direction of the prongs, the second taper has a shorter length than the first taper has. Providing the tapered end part with an additional taper—the second taper—having a taper direction different than the direction of first taper and a taper length smaller than the taper length of the main taper, additionally sharpens the sharp tip without basically decreasing the strength of the sharp tip. In this application a taper direction is understood to be a direction, transverse to the length direction of the prong, in which the cross-section reduces due to the respective taper. According to one example of this embodiment, the first taper direction is, when in the closed position, towards the associated supporting area and the second taper direction is away from the associated supporting area. According to another alternative or supplementary example of this embodiment the first taper direction the first taper direction and second taper direction are at an angle with respect to each other, such as perpendicular to each other.

According to a further embodiment of the invention, the tapered end parts of the prongs of the first fork each have a ring facing surface, which, in the closed position, faces the associated supporting area; and, in the closed position, the ring facing surface or at least a substantial part of the ring facing surface of each said prong of the first fork lies parallel to and optionally against the associated supporting area (with optionally vessel wall in between the ring facing surface and the ring structure when the anastomosis is established). In this case ‘substantial part’ is to be understood as at least 75%, such as at least 80% or 85%, of the ring facing surface. Ensuring the ring facing surface and associated supporting area being parallel for at least a substantial part ensures the tapered ends being well supported and well prevented from being deformed/bended.

According to a further embodiment of the invention, the supporting areas are recessed in the prong end side of the ring structure. The supporting areas being recessed, provides lateral support—in a direction parallel to the ring surface—to the tapered end parts.

According to a further embodiment of the invention, the clip may have a second two prong fork for attachment to the second vessel of the anastomosis, the first two prong fork being for attachment to the first vessel of the anastomosis. In this embodiment, the clip further comprises:
a second two prong fork having a fork base and two prongs extending from the fork base to a free end part, the free end parts of the prongs of the second fork tapering to a sharp tip,
a further clamping structure configured to clamp the second fork in the closed position against the ring structure of first fork,
   wherein the ring structure and second fork are movable with respect to each other between an open position and the closed position;
   wherein, in the open position, the ring structure and second fork are arranged at a distance from each other;
   wherein, in the closed position and viewed with respect to/in the ring plane:
the first fork is arranged at one side of the ring plane and the second fork is arranged at the other side of the ring plane;
the prongs of the second fork extend along the ring plane, and
the fork base of the second fork is located at the base side of the ring structure whilst the free end parts of the prongs of the second fork are located at the prong end side of the ring structure;
the free end parts of the prongs of the second fork lie submerged or recessed in an inner space bounded by the ring structure such that in the closed position the sharp tips of the second fork are shielded by the ring structure.
   Although this embodiment has a second prong with sharp tips, damaging vessel walls and surrounding tissue is prevented by having the free end parts of the second prongs—which carry the sharp tips—are submerged in the inner space bounded by the ring structure.

According to a further embodiment of the invention with a second fork, the second fork is, in the closed position, clamped against the first fork with a second clamping force, the second clamping force being directed opposite to the first clamping force, and the second clamping force being smaller than the first clamping force. The second clamping force being smaller than the first clamping force, prevents the first prongs of the first fork from being loosened or released with respect to the ring structure.

According to a further embodiment of the invention, the ring structure further has a left side part connecting the base side with the prong end side, and, opposite to the left side part, a right side part connecting the base side with the prong end side.

According to a further embodiment of the invention with left and right side part, the left side part and right side part have, viewed in radial direction of the ring structure a radial thickness, and is said length of the supporting areas larger than the radial thickness, such as at least 1.5× the radial thickness, such as at least 1.75× larger than the radial thickness (RT) or at least 2× larger than the radial thickness (RT).

According to a further embodiment of the invention with left and right side part, a section of a right one of said prongs of the first fork extends, in the closed position, parallel and/or adjacent to the right side part of the ring structure, whilst a section of a left one of said prongs of the first fork extends parallel and/or adjacent to the left side part of the ring structure.

According to a further embodiment of the invention with left and right side part, said section of the right prong of the first fork and said section of the left prong of the first fork lie, in the closed position, submerged or recessed in the inner space bounded by the ring structure.

According to a further embodiment of the invention with left and right side part, a section of a right one of the prongs of the second fork extends, in the closed position, parallel and/or adjacent to the right side part of the ring structure, whilst a section of a left one of the prongs of the second fork extends parallel and/or adjacent to the left side part of the ring structure.

According to a further embodiment of the invention with left and right side part, said section of the right prong of the second fork and said section of the left prong of the second fork lie, in the closed position, submerged or recessed in the inner space bounded by the ring structure.

### BRIEF DESCRIPTION OF THE DRAWING

The invention will be explained further with reference to the drawings. In these drawings:

FIG. 1 shows schematically a first exemplary embodiment of the anastomosis clip according to the invention, in which the anastomosis clip has one ‘two prong’ fork, in this figure:

FIG. 1A shows, in side view, the anastomosis clip with the (first) two prong fork and the rings structure as two separate parts and in open position;

FIG. 1B shows, in side view, the anastomosis clip of FIG. 1A with the two separate parts assemble together and still in open position;

FIG. 1C shows, in side view, the anastomosis clip of FIG. 1B in the closed position;

FIG. 1D shows a bottom view according to arrow I—see FIG. 1C—onto the anastomosis clip of FIG. 1;

FIG. 1E shows a bottom view according to arrow I—see FIG. 1C—onto the ring structure of the anastomosis clip of FIG. 1; and

FIG. 1F shows a bottom view according to arrow I—see FIG. 1C—onto the (first) two prong fork of the anastomosis clip of FIG. 1.

FIG. 2 shows schematically a second exemplary embodiment of the anastomosis clip according to the invention, in which the anastomosis clip has two ‘two prong’ forks, in this figure:

FIG. 2A shows, in side view, the anastomosis clip with the first and second two prong fork and the rings structure in open position;

FIG. 2B shows, in side view, the anastomosis clip of FIG. 2A with the first and second two prong fork and the rings structure in closed position;

FIG. 2C shows a top view according to arrow II—see FIG. 2B—onto the anastomosis clip of FIG. 2B;

FIG. 2D shows a bottom view according to arrow III—see FIG. 2B—onto the anastomosis clip of FIG. 2B;

FIG. 2E shows a top view according to arrow II—see FIG. 2B—onto the ring structure of the anastomosis clip of FIG. 2;

FIG. 2F shows a side view according to arrow IV—see FIG. 2C—onto the ring structure of the anastomosis clip of FIG. 2;

FIG. 2G shows a top view according to arrow II—see FIG. 2B—onto the first two prong fork of the anastomosis clip of FIG. 2;

FIG. 2H shows a side view according to arrow IV—see FIG. 2C—onto the first two prong fork of the anastomosis clip of FIG. 2, in FIG. 2H:

FIG. 2H-1 is a side view of the complete first fork, and

FIG. 2H-2 is an enlarged side view of a detail of the first fork, the detail being the distal end of a prong of the first fork;

FIG. 2I shows a top view according to arrow II—see FIG. 2B—onto the second two prong fork of the anastomosis clip of FIG. 2; and

FIG. 2J shows a side view according to arrow IV—see FIG. 2C—onto the second two prong fork of the anastomosis clip of FIG. 2.

FIG. 3 shows a schematic perspective view of the ring structure according to FIG. 1 inserted into a vessel according to a first example;

FIG. 4 shows a schematic perspective view of the ring structure according to FIG. 1 inserted into a vessel according to a second example;

FIG. 5 shows a schematic perspective view of the ring structure according to FIG. 1 inserted into a vessel according to a third example;

FIG. 6 shows a schematic perspective view of the first two prong fork according to FIG. 1 inserted into a vessel according to a third example. In this figure:

FIG. 6A shows the first two prong fork in a first position relative to the vessel; and

FIG. 6B shows the first two prong fork in a second position relative to the vessel.

### DETAILED DESCRIPTION OF THE EMBODIMENTS

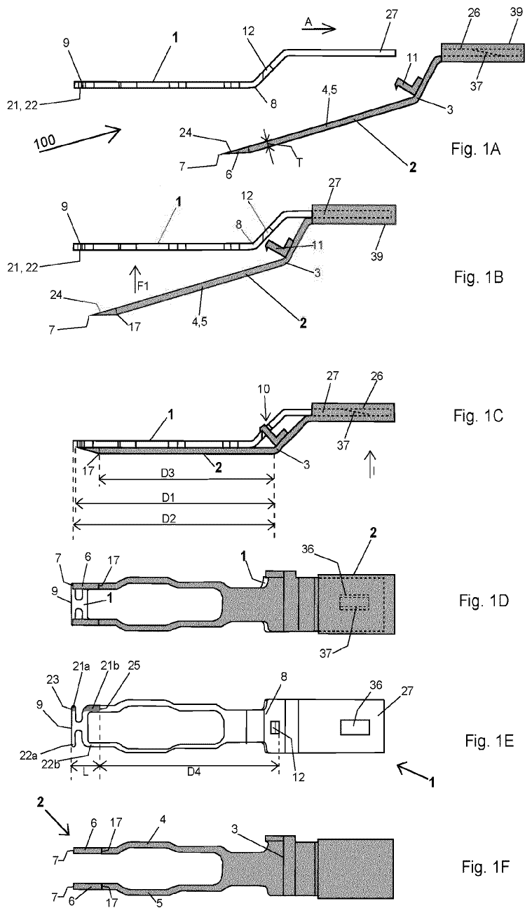

FIG. 1 shows, as an example of the invention, a first exemplary embodiment 100 of an anastomosis clip 100 according to the invention in several views, namely FIG. 1A to FIG. 1F.

The anastomosis clip according to FIG. 1 comprises, as a first component, a first two prong fork 2—shown in grey—having a fork base 3 and two prongs 4, 5 extending from the fork base 3 to a tapered end part 6. The taper of the tapered end part 6 essentially begins at a taper base 17. The tapered end parts 6 tapers from a taper base 17 to a sharp tip 7.

The anastomosis clip according to FIG. 1 further comprises, as a second component, a ring structure 1—shown in white—which defines a ring plane. The ring plane essentially is the plane in which the ring extends. The ring plane may be a flat plane or a curved plane.

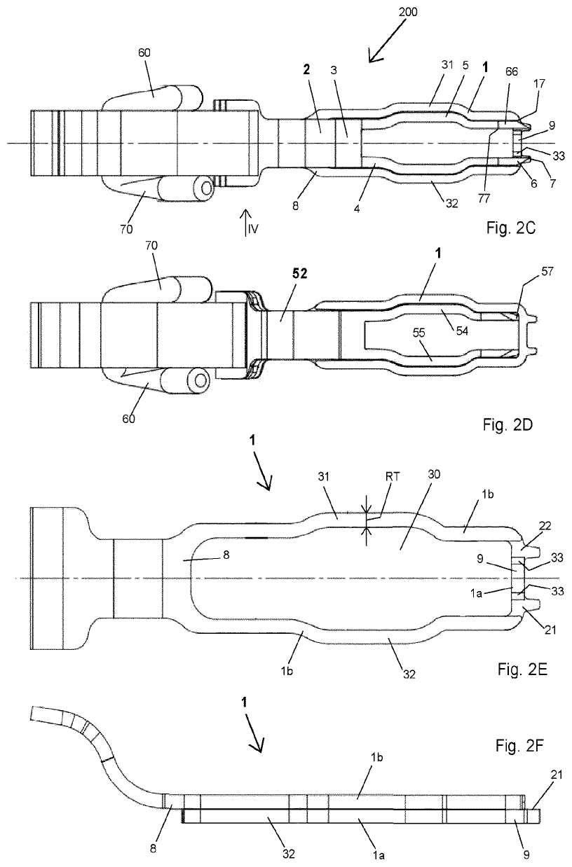

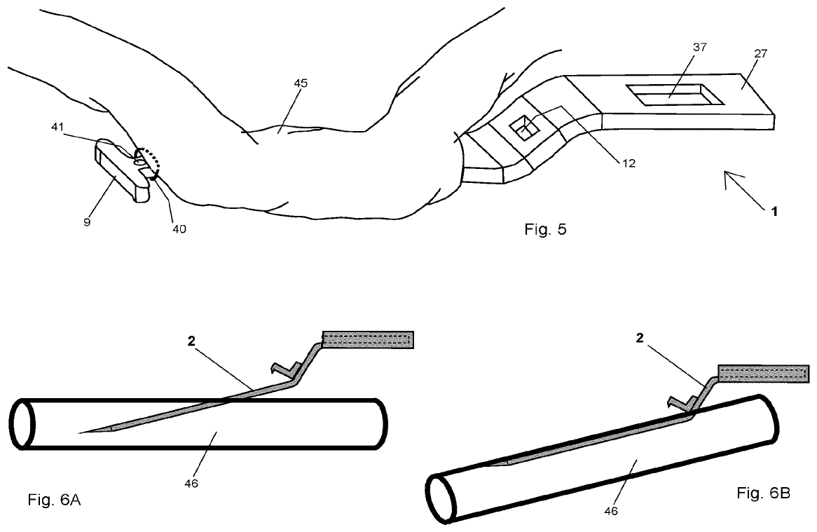

The anastomosis clip according to FIG. 1 further comprises, as a third component, a clamping structure 10 configured to clamp the first fork 2 in a closed position with a first clamping force towards the ring structure 1. Without any vessel, the clamping structure may, when in the closed position, clamp the first fork 2 directly against the ring structure 1. In applied condition, the clamping structure may, when in the closed position, clamp the first fork 2 indirectly against the ring structure 1 with one or two vessel wall between the first fork 2 and the ring structure 1, see for example FIGS. 3-5 in combination with FIG. 6.

The ring structure 1 and the first fork 2 are movable with respect to each other from an open position—see FIG. 1B and also FIG. 1A—to the closed position—see FIG. 1C.

As can be seen in FIGS. 1A and 1B, the ring structure 1 and first two prong fork 2 of the anastomosis clip according to the (first exemplary embodiment 100 of the) invention may made available to the surgeon as—case a)—two separate parts—see FIG. 1A—or as—case b)—one unit.

In the case of a), the surgeon may first attach the ring structure 1 to one of the two anastomosis vessels—see FIGS. 3-5 and attach the two prong fork 2 to the other of the two anastomosis vessels—see FIG. 6—and then join the ring structure 1 and the first two prong fork 2 to one unit. This joining may be accomplished n several manners. According to one example, this joining is accomplished by inserting the proximal end 27 of the ring structure 1 into a recess or slit 26 provided at the proximal end 39 of the first fork 2. According to this example, the ring structure 1 and the first two prong fork 2 may be fixed relative to each other by means of a resilient lip 37 or rib provided in the proximal end 39 of the first fork 1, which resilient lip 37 engages in a recess 36—see FIG. 1E—provided in the proximal end 27 of the first fork, or—not shown—the other way around by means of a resilient lip or rib provided on the proximal end 27 of the ring structure 1, which resilient lip/rib engages in a recess provided in the recess/slit 26 in the proximal end 39 of the first fork 2. The joining of the first fork with the ring structure to form one unit may also be accomplished in other manners. This joining of the first fork 2 with the ring structure 1 may result in the condition as shown in FIG. 1B. As will be clear, the recess/slit 26 and lip 37 may alternatively be provided at the proximal end 27 of the ring structure 1.

In the case of b), the first fork 2 and ring structure 1 are made available to the surgeon as one unit, for example a pre-fab unit. In the case of b), the recess/slit 26, and/or the lip/rib 37, and/or the recess 36 may be absent. The first fork 2 and ring structure 1 may for example be welded, glued, bolted, or screwed to each other. But any other technique of joining the first fork 2 and ring structure 1 may be used as well.

As can be seen in both FIG. 1A and FIG. 1B, the ring structure 1 and the first fork 2 are arranged at a distance from each other in the open position.

FIG. 1C shows the closed position. As can be seen this FIG. 1C, the prongs 4, 5 of the first fork extend, in the closed position, along the ring plane, and the fork base 3 of the first fork 2 is located at a base side 8 of the ring structure 1 whilst the tapered end parts 6 of the prongs 4, 5 of the first fork 2 are located at a prong end side 9 of the ring structure 1, the prong end side 9 being, viewed along the ring plane, opposite the base side 8.

Referring to FIG. 1E, the ring structure 1 has, for each of said prongs 4,5 of the first fork 2, a first supporting area 21 associated to the first prong 4 and a second support area 22 associated to the second prong 5. In the example of FIG. 1E, the first supporting area 21 is shown in grey to clarify the supporting area. This grey has been omitted with respect to the second support area. As can further be seen in the example of FIG. 1E, the first and second support area each consist of two sub-areas 21a, 21b respectively 33a, 22b, which sub-areas are separated by an intermediate space. It will however be understood that each support area may also consist of a single area, or three or more sub-areas.

Referring to FIG. 1E, each said supporting area 21, 22 has, viewed with respect to the fork base 3 of the first fork 2, a distal end 23 and a proximal end 25. Further, as can be seen in FIG. 1 in general, each supporting area 21, 22 is configured to support a part of the associated prong when the first fork is, in the closed position, clamped with the first clamping force towards the ring structure.

Referring to FIG. 1C, a first distance D1—measured from the fork base 3 of the first fork 2 to the sharp tip 7 of the tapered end part of a said prong 4,5—is, in the closed position, at most as long as a second distance D2—measured from the fork base 3 of the first fork 2 to the distal end of the associated supporting area 21, 22—. This results in that, viewed in a transverse direction transverse to the ring plane, each said sharp tip 7 is shielded by its associated supporting area 21 or 22.

Referring to FIGS. 1C and 1E, a third distance D3—measured from the fork base 3 of the first fork 2 to the taper base 17 of the tapered end part of a said prong 4,5—is at least as long as a fourth distance D4—measured from the fork base (3) of the first fork (2) to the proximal end (25) of the associated supporting area (21, 22)—. This results in that, viewed in the transverse direction, each said taper base 17 is supported by its associated supporting area 231 or 22.

As can be seen in FIG. 1—i.e. the FIGS. 1A-1F—the clamping structure 10 may—for example—comprise a passage 12 provided in the ring structure 1 and a snap member 11 provided on the first fork 2. As will be understood this may also be the other way around—i.e. the passage 12 provided in the first fork 1 and the snap member 11 being provided on the ring structure. More in general the clamping structure may be provided in other manners as well.

Referring to FIG. 1E, the supporting areas 21, 22 have, viewed from their proximal end 25 to their distal end 23, an area length L. This area length L may be equal to or larger than the length of the tapered end part 6. The area length L may according to an embodiment be at least 110% of the length of the tapered end part, for example at least 115% of the length of the tapered end part 6 or at least 120% of the length of the tapered end part 6, or at least 125% of the length of the tapered end part 6. In addition or alternatively, the area length L may, according to a further respectively alternative embodiment, be is at least 2× the thickness T—see FIG. 1A—of the prongs 4, 5 of the first fork 2, for example at least 2.5× of the thickness T of the prongs 4, 5 of the first fork 2, or at least 3.0× the thickness T of the prongs 4, 5 of the first fork 2. According to a further additional or alternative embodiment, wherein the area length L may be at most 6× the thickness T of the prongs 4, 5 of the first fork 2, for example at most 5× the thickness T of the prongs 4, 5 of the first fork 2.

Referring to FIG. 1B, the tapered end parts 6 of the prongs 4, 5 of the first fork 2 each have a ring facing surface 24, which, in the closed position, faces the associated supporting area 21, 22. As can be seen in FIG. 1C, the ring facing surface 24 or at least part of the ring facing surface 24 of each said prong 4, 5 of the first fork 2 lies, in the closed position, parallel to and optionally against the associated supporting area 21, 22.

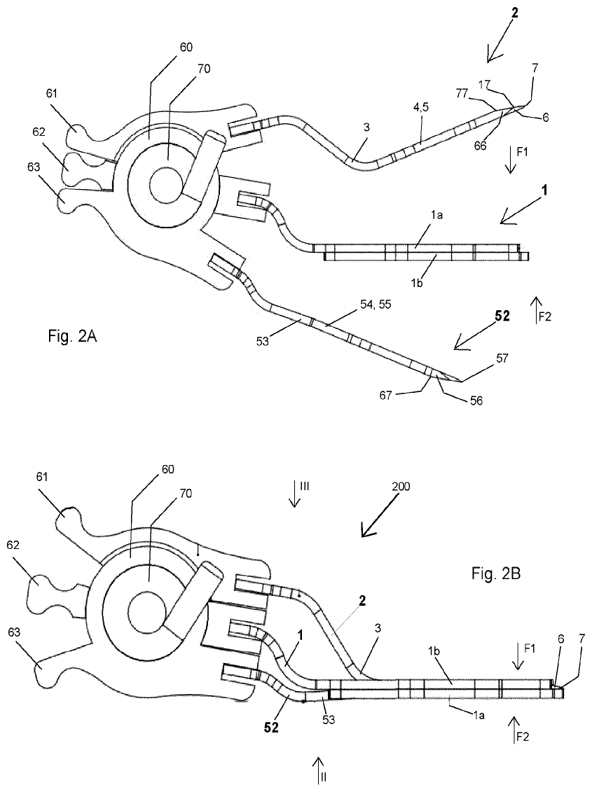

FIG. 2 shows, as an example of the invention, a second exemplary embodiment 200 of an anastomosis clip according to the invention in several views, namely FIG. 2A to FIG. 2J.

Like the first exemplary embodiment 100 of FIG. 1, the anastomosis clip according to FIG. 2 comprises, as a first component, a first two prong fork 2 having a fork base 3 and two prongs 4, 5 extending from the fork base 3 to a tapered end part 6. The taper of the tapered end part 6 essentially begins at a taper base 17. The tapered end parts 6 tapers from a taper base 17 to a sharp tip 7.

Like the first exemplary embodiment 100 of FIG. 1, the anastomosis clip according to FIG. 2 further comprises, as a second component, a ring structure 1 which defines a ring plane. The ring plane essentially is the plane in which the ring extends. The ring plane may be a flat plane or a curved plane.

Like the first exemplary embodiment 100 of FIG. 1, the anastomosis clip according to FIG. 2 further comprises, as a third component, a clamping structure 60 configured to clamp the first fork 2 in a closed position with a first clamping force towards the ring structure 1. Without any vessel, the clamping structure may, when in the closed position, clamp the first fork 2 directly against the ring structure 1. In applied condition, the clamping structure may, when in the closed position, clamp the first fork 2 indirectly against the ring structure 1 with one or two vessel wall between the first fork 1 and the ring structure 2, see for example FIGS. 3-5 in combination with FIG. 6.

In addition to the first exemplary embodiment 100 of FIG. 1, the anastomosis clip according to the exemplary embodiment of FIG. 2 further comprises, as a fourth component, a second two prong fork 52 having a fork base 53 and two prongs 54, 55 extending from the fork base 53 to a tapered end part 56. The taper of the tapered end part 56 essentially begins at a taper base 67. The tapered end parts 56 taper from a taper base 67 to a sharp tip 57.

In addition to the first exemplary embodiment 100 of FIG. 1, the anastomosis clip according to the exemplary embodiment of FIG. 2 further comprises, as a fifth component, a further clamping structure 70 configured to clamp the second fork 52 in a closed position with a second clamping force towards the ring structure 1. Without any vessel, the further clamping structure 70 may, when in the closed position, clamp the second fork 2 directly against the ring structure 1. In applied condition, the further clamping structure 70 may, when in the closed position, clamp the second fork 52 indirectly against the ring structure 1 with one or two vessel wall between the second fork 52 and the ring structure 2. This is similar as is shown in relation to the first exemplary embodiment 100 in the example of FIGS. 3-5 in combination with FIG. 6.

The ring structure 1 and the first fork 2 are movable with respect to each other from an open position—see FIG. 2A—to a closed position—see FIG. 2B. Also the ring structure 1 and the second fork 52 are movable with respect to each other from an open position—see FIG. 2A—to a closed position—see FIG. 2C. The first fork 2 and second fork 52 may move independently from each other with respect to the first ring structure 1 or may move simultaneously—like partly simultaneously—with respect to the ring structure. As can be seen in FIG. 1A, the first fork 2 and second fork 52 can be brought in the open position with respect to the ring structure 1 by pushing the grips 61 and 63 towards central part/grip 62. The pushing of the grip 61 towards the grip 62 may be against a first resilient force exerted by a spring 60, and pushing of the grip 63 towards the grip 62 may be against a second resilient force exerted by a spring 70. The grips 61 and 63 may be pushed simultaneously or separately from each other towards the central part/grip 62.

It is to be noted, that the clamping construction 60, 70 of the second exemplary embodiment 200 may also be configured differently. It is for example conceivable that these clamping constructions 60, 70 are configured like the clamping construction 10 according to the exemplary embodiment of FIG. 1. In this case, the proximal end of the ring structure 1 may for example be provided with a body having a first slit 26 for receiving the proximal end of the fork 2 and a similar second slit for receiving the proximal end of the second fork second fork 52. To ensure locking, the proximal end of either the second fork 52 or the ring structure 1 may, like in the first exemplary embodiment 100, be provided with a lip/rib, similar to lip/rib 37. It is also conceivable that—similar to the first exemplary embodiment 100—the ring structure 1, the first fork 2, and second fork 3 are made available to the surgeon as one unit. To provide the clamping structures, both the first fork 2 and second fork 52 may be provided with a snap member 11 cooperating with a snap member 12 provided on the ring structure.

Similar as in the first exemplary embodiment 100, not only the ring structure 1 and the first fork 2 are movable with respect to each other between an open position and a closed position, but also the ring structure 1 and second fork 52 are movable with respect to each other between an open position and a closed position.

Further, similar as in the first exemplary embodiment 100, not only the ring structure 1 and the first fork 2 are arranged at a distance from each other in the open position, but also the ring structure 1 and the second fork 52 are arranged at a distance from each other in the second position.

With the second exemplary embodiment 200 in the closed position, the first fork 2 is arranged at one side of the ring plane and the second fork 52 is arranged at the other side of the ring plane. With the second exemplary embodiment 200 in the closed position, the fork base 53 of the second fork 52 is located at the base side 8 of the ring structure whilst the free end parts 56 of the prongs 54, 55 of the second fork 52 are located at the prong end side 9 of the ring structure 1.

Although, the tapered end parts 56 of the second fork 52 may, in the closed position, similarly like the tapered end parts of the first fork 2 be supported by an associated supporting area provided on the ring structure 1 with the sharp tips 57 being shielded by the associated supporting area provided on the ring structure 1, surrounding tissue is according to the second exemplary embodiment 200, in the closed position, shielded from the sharp tips 57 due to the free end parts 56 of the prongs 54, 55 lying submerged in an inner space 30 bounded by the ring structure 1.

In this second exemplary embodiment 200, it is according to a further elaboration conceivable that the first fork 2 is provided with associated support areas supporting the second fork 52 in its closed position.

In this second exemplary embodiment 200, it is according to an additional or alternative further elaboration conceivable that, in the closed position, the second fork 52 is clamped against the first fork 2 with a second clamping force F2, the second clamping force F2 being directed opposite to the first clamping force F1, and the second clamping force F2 being smaller than the first clamping force F1. The second clamping force F2 being smaller than the first clamping force F1, prevents the second fork 52 from lifting the first fork 2 towards the open position of the first fork.

Referring to FIG. 2E, the ring structure 1—according to not only the second exemplary embodiment but also according the first exemplary embodiment—further have a left side part 31 connecting the base side 8 with the prong end side 9, and, opposite to the left side part 31, a right side part 32 connecting the base side 8 with the prong end side 9.

Further, referring to FIG. 2E, the ring structure 1—according to not only the second exemplary embodiment but also according the first exemplary embodiment—the left side part 31 and right side part 32 may optionally have, viewed in radial direction of the ring structure 1, a radial thickness RT, whilst the length L of the supporting areas 21, 22 is larger than the radial thickness RT, such as at least 2× larger than the radial thickness RT. Experimental practise shows that when the length L of the supporting areas 21, 22 is longer than the radial thickness, especially at least twice as long, the tapered end parts 6 are quite well prevented from bending when in the closed position.

As can be seen in FIG. 1D, and FIG. 2C, a section of a right one 4 of said prongs of the first fork 2 may, according to a further embodiment, extend, in the closed position, parallel to the right side part 32 of the ring structure 1, whilst a section of a left one 5 of said prongs of the first fork may extend parallel to the left side part 31 of the ring structure 1. Referring to FIG. 2D, a section of a right one 54 of the prongs of the second fork 52 may—in the closed position—according to a further embodiment similarly extend parallel to the right side part 32 of the ring structure 1, whilst a section of a left one 55 of the prongs of the second fork 52 may extend parallel to the left side part 31 of the ring structure 1.

As is shown in FIG. 2C and FIG. 2B, the section of the right prong 4 which is in the closed position parallel to the right side of the ring structure and the section of the left prong 5 which is in the closed position parallel to the left side of the ring structure 1 may, optionally, lie submerged in the inner space 30 bounded by the ring structure 1. This not only applies for the second exemplary embodiment 200 but also to other embodiment of the invention, like the first exemplary embodiment 100. Referring to FIG. 2C and FIG. 2E ribs 33 may be provided adjacent the supporting areas 22, 24 such that the tapered end parts lie so to say in recesses. This prevents the tapered end parts 6 of the prongs 4, 5 from moving in sideward direction, when in the closed position.

As is shown in FIG. 2D and FIG. 2B, the section of the right prong 54 which is in the closed position parallel to the right side of the ring structure 1 and the section of the left prong 55 which is in the closed position parallel to the ring structure 1 may, optionally, lie submerged in the inner space 30 bounded by the ring structure 1.

As can be seen in FIG. 2A, FIG. 2C, FIG. 2G, FIGS. 2H-1 and 2H-2, the tapered end parts 6 of the prongs 4, 5 of the first fork 2 may—according to a further embodiment—each have a three tapers. See also the enlarged view of a detail in FIG. 2H-2. The tapered end has a main taper 67 starting at 77, an additional taper 35 having its taper base at 17, and another additional taper 38 starting at 87. As can be seen in FIG. 2C, the additional taper 35 having its taper base at 17 is about fully overlapped by the associated supporting area 21, 22. In one embodiment, the part of the prong with the additional taper 35 may be seen as the tapered end part 6. Further it can be seen in FIG. 2C that about 40%-50% of the main taper 67 overlaps with an associated supporting area 21, 22. In another embodiment of the invention, the part of the prong with the additional taper 67 may be seen as the tapered end part 66.

The main taper 67 has a main taper direction S1 and one or more further tapers 35, 38 have a further taper direction S2, S3—see also the enlarged detail of the distal end of the prong 4 shown in FIG. 2H-2—. The main taper direction S1 is different from the further taper directions S2, S3. Viewed in length direction of the prongs, the further tapers 35, 38 have a shorter length than the main taper 67 has. Further, additional taper direction S2 of the taper 35 is different from the additional taper directions S3 of the taper 38. Viewed in length direction of the prongs, the further taper 38 has a shorter length than the further taper 35 has. Although this is shown in relation to the second exemplary embodiment 200, it will be clear that this can also be the case in other embodiments of the invention, such as the first exemplary embodiment.

Referring to FIG. 2H, the direction of the main taper 67—indicated by arrow S1 in FIG. 2H-2—may be towards the associated supporting area and the direction of the additional taper 38—indicated by arrow S2 in FIG. 2H-2—may be in a direction away from the associated supporting area. The additional taper 38 allows the sharp tip 57 sharpening in a shorter prong length. Depending on material of the prongs, thickness of the prongs and clamping force, the risk of bending/deforming of the tapered end part may essentially be present in the further tapered part 35 and not already at zone where the main taper 67 starts.

Referring to FIGS. 2G and 2H-2, the direction of the main taper 6 and the direction of the further tapers 35 may additionally or alternatively also be at an angle with respect to each other, such as perpendicular to each other—as is indicated by means of the rear view on arrow S3, which arrow is perpendicular to the plane of drawing pointing towards the plane of drawing.

FIGS. 3-6 show examples of the use of first exemplary embodiment 100. FIGS. 3-5 differ in how the vessel is attached to the ring structure 1.

In FIG. 3, the ring structure 1 is mounted to the open end of vessel 43 with the distal end of the ring structure so to say inside the open end of the vessel 43. The edge of the vessel wall around the open end of the vessel 43 may for example be glued or sutured to the ring structure to provide a seal all around. The vessel 43 may however also be attached in another manner to the ring structure. The vessel 43 may be a natural vessel or an artificial vessel. In case of an artificial vessel the open end of the vessel 43 may be attached along the ring of the ring structure by a wide range of mounting techniques. It is also conceivable to integrate the ring of the ring structure 1 with or in the end of an artificial vessel 43.

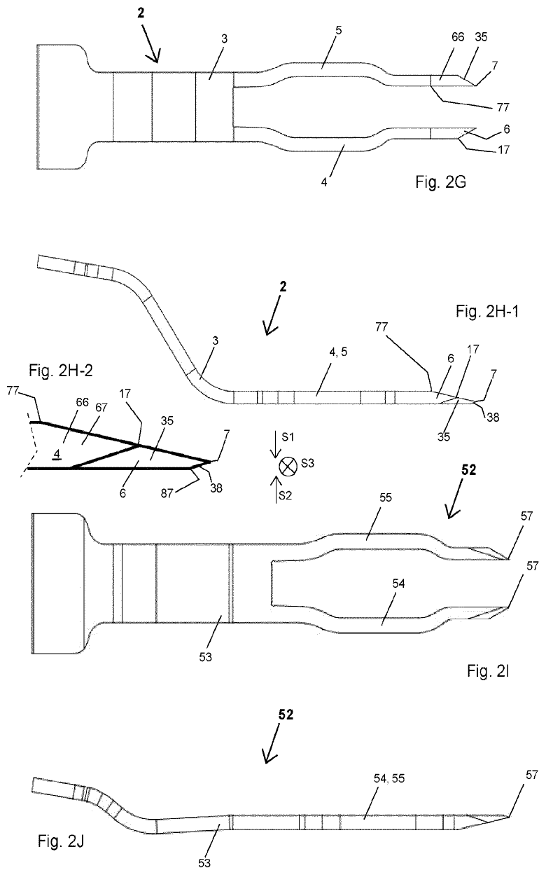

In FIG. 4, the ring structure 1 is mounted to the open end of vessel 44 with the distal end of the ring structure so to say projecting beyond the open end of the vessel 44. As shown, a suture 40 may be used to attach the vessel end to the ring structure. For this purpose one or more bores 41 may be provided in the ring structure, for example a plurality of bores 41 distributed along the circumference of the ring of the ring structure. A glue/adhesive may be used to ensure good sealing all around the ring of the ring structure. The vessel 43 may be a natural vessel or an artificial vessel.

In FIG. 3 an FIG. 4 the vessel 43 respectively vessel 44 are the T-part of an ETS-anastomosis or an ETE-anastomosis.

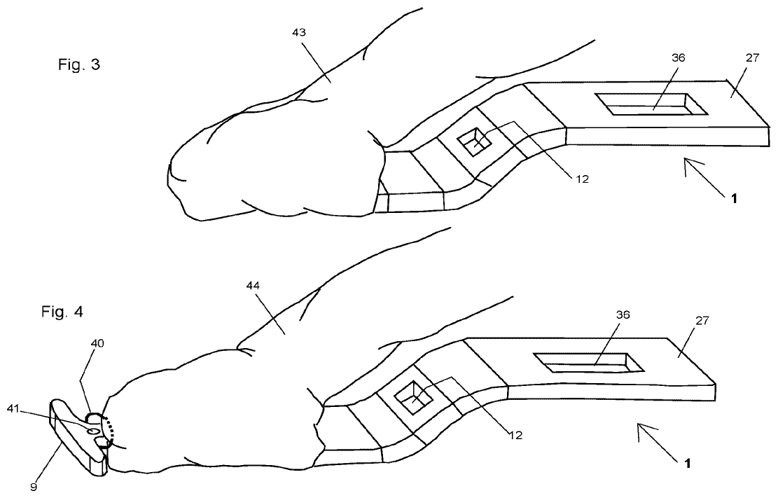

In FIG. 5 the vessel 45 is one of the S-parts of a STS-anastomosis. In this example, the sidewall of the vessel 45 will be provided with an aperture surrounding the ring of the ring structure. Like in FIG. 3 and FIG. 4 the vessel 45 may be a natural vessel or an artificial vessel. Further like in FIGS. 3 and 4, the vessel may be attached to the ring structure by means of glue/adhesive and/or suture and/or other manners f attachment.

In FIG. 6 it is shown how the first fork 2 is mounted to the vessel 46. The sharp tips 7 are pierced through the wall of vessel 46—see FIG. 6A—and subsequently the prongs of the forks are brought in a position parallel to the wall of the vessel 46.

As will be clear to the skilled man, the first fork and second fork may be provided to the surgeon as one unit or—as is shown in FIG. 3-6—two parts to be mounted together by inserting the distal end 27 of the ring structure 1 into the slit 26 in the distal end of the first fork.

As will further be clear to the skilled man, the vessel 46 of FIG. 5 may also be mounted on a second fork 52 in a manner similar to what is shown in FIG. 6, resulting in an anastomosis clip according to the second exemplary embodiment of the invention.

Embodiments and further embodiments of the present invention may be expressed in words as set out in the following clauses:

Clause 1] An anastomosis clip (100; 200) for connecting a first vessel to the side wall of a second vessel,
wherein the anastomosis clip comprises:
   a first two prong fork (2) having a fork base (3) and two prongs (4, 5) extending from the fork base (3) to a tapered end part (6), the tapered end parts (6) tapering from a taper base (17) to a sharp tip (7),
   a ring structure (1) defining a ring plane, and
   a clamping structure (10; 60) configured to clamp the first fork (2), in a closed position, with a first clamping force towards the ring structure (1);
wherein the ring structure (1) and the first fork (2) are movable with respect to each other from an open position to the closed position;
wherein, in the open position, the ring structure (1) and the first fork (2) are arranged at a distance from each other;
wherein, in the closed position:
   the prongs (4, 5) of the first fork extend along the ring plane, and
   the fork base (3) of the first fork (2) is located at a base side (8) of the ring structure (1) whilst the tapered end parts (6) of the prongs (4, 5) of the first fork (2) are located at a prong end side (9) of the ring structure (1), the prong end side (9) being, viewed along the ring plane, opposite the base side (8);
wherein the ring structure (1) has, for each of said prongs (4,5) of the first fork (2), an associated supporting area (21, 22) associated to a said prong (4, 5) of the first fork (2);
wherein each said supporting area (21, 22):
   has, viewed with respect to the fork base (3) of the first fork (2), a distal end (23) and a proximal end (25), and
   is configured to support a part of the associated prong when the first fork is, in the closed position, clamped with the first clamping force towards the ring structure; and
wherein, in the closed position a first distance (D1) from the fork base (3) of the first fork (2) to the sharp tip (7) of the tapered end part of a said prong (4,5) is at most as long as a second distance (D2) from the fork base (3) of the first fork (2) to the distal end of the associated supporting area (21, 22) such that, viewed in a transverse direction transverse to the ring plane, each said sharp tip is shielded by its associated supporting area.

Clause 2] An anastomosis clip (100, 200) according to clause 1, wherein, in the closed position, the associated supporting area overlaps at least about 35%, such as at least about 50%, of the tapered end part.

Clause 3] The anastomosis clip (100, 200) according to clause 1 or 2, wherein the supporting areas have, viewed from their proximal end to their distal end, an area length (L).

Clause 4] The anastomosis clip (100, 200) according to clause 3, wherein the area length (L) is at least 1.5×, such as at least 2.0× or at least 2.5×, the thickness (T) of the prongs (4, 5) of the first fork (2).

Clause 5] The anastomosis clip (100, 200) according to one of the clauses 3-4, wherein the area length (L) is at most 6×, such as at most 5×, the thickness (T) of the prongs (4, 5) of the first fork (2).

Clause 6] The anastomosis clip (100, 200) according to one of the clauses 3-5, wherein the area length (L) is equal to or larger than the length of the tapered end part.

Clause 7] The anastomosis clip (100, 200) according to one of the clauses 3-6, wherein the area length (L) is at least 110%, such as at least 115% or 120% or 125%, of the length of the tapered end part.

Clause 8] The anastomosis clip (100, 200) according to one of the proceeding clauses, wherein, in the closed position, a third distance (D3) from the fork base (3) of the first fork (2) to the taper base (17) of the tapered end part of a said prong (4,5) is at least as long as a fourth distance (D4) from the fork base (3) of the first fork (2) to the proximal end (25) of the associated supporting area (21, 22) such that, viewed in the transverse direction, each said taper base (17) is supported by its associated supporting area.

Clause 9] The anastomosis clip (100, 200) according to one of the preceding clauses, wherein the tapered end parts (6) of the prongs (4, 5) of the first fork (2) each have a first taper with a first taper direction (S1) and a second taper with a second taper direction (S2, S3); wherein the first taper direction (S1) is different from the second taper direction (S2, S3); and wherein, viewed in length direction of the prongs (4, 5), the second taper has a shorter length than the first taper has.

Clause 10] The anastomosis clip according to clause 9, wherein, viewed in the closed position, the first taper direction (S1) is towards the associated supporting area (21, 22) and the second taper direction (S2) is away from the associated supporting area (21, 22).

Clause 11] The anastomosis clip according to clause 9 or 10, wherein the first taper direction (S1) and second taper direction (S3) are at an angle with respect to each other, such as perpendicular to each other.

Clause 12] The anastomosis clip (100, 200) according to one of the preceding clauses, wherein the tapered end parts (6) of the prongs (4, 5) of the first fork (2) each have a ring facing surface (24), which, in the closed position, faces the associated supporting area (21, 22); and
wherein, in the closed position, the ring facing surface (24) or at least part of the ring facing surface (24) of each said prong (4, 5) of the first fork (2) lies parallel to and optionally against the associated supporting area (21, 22).

Clause 13] The anastomosis clip (100, 200) according to one of the preceding clauses, wherein the supporting areas (21, 22) are recessed in the prong end side (9) of the ring structure (1).

Clause 14] The anastomosis clip (200) according to one of the preceding clauses, wherein the clip (200) further comprises:
a second two prong fork (52) having a fork base (53) and two prongs (54, 55) extending from the fork base (53) to a free end part (56), the free end parts (56) of the prongs (54, 55) of the second fork (52) tapering to a sharp tip (57),
a further clamping structure (70) configured to clamp the second fork (52), in a closed position towards the ring structure (1) of first fork (2),
   wherein the ring structure (1) and second fork (52) are movable with respect to each other between an open position and the closed position;
   wherein, in the open position, the ring structure (1) and second fork (52) are arranged at a distance from each other;
   wherein, in the closed position and viewed with respect to the ring plane:
the first fork (2) is arranged at one side of the ring plane and the second fork (52) is arranged at the other side of the ring plane;
the prongs (54, 55) of the second fork extend along the ring plane, and
the fork base (53) of the second fork (52) is located at the base side (8) of the ring structure (1) whilst the free end parts (56) of the prongs (54, 55) of the second fork (52) are located at the prong end side (9) of the ring structure (1);
the free end parts (56) of the prongs (54, 55) of the second fork (52) lie submerged in an inner space (30) bounded by the ring structure (1) such that, in the closed position, the sharp tips (56) of the second fork (52) are shielded by the ring structure (1).

Clause 15] The anastomosis clip (200) according to clause 14,
wherein, in the closed position, the second fork (52) is clamped towards the first fork (2) with a second clamping force (F2), the second clamping force (F2) being directed opposite to the first clamping force (F1); and
wherein the second clamping force (F2) is smaller than the first clamping force (F1).

Clause 16] The anastomosis clip (100, 200) according to one of the preceding clauses,
wherein the ring structure (1) further has a left side part (31) connecting the base side (8) with the prong end side (9), and, opposite to the left side part (31), a right side part (32) connecting the base side (8) with the prong end side (9).

Clause 17] The anastomosis clip (100, 200) according to clause 16,
wherein the left side part (31) and right side part (32) have, viewed in radial direction of the ring structure (1), a radial thickness (RT), and
wherein said length (L) of the supporting areas (21, 22) is larger than the radial thickness (RT), such as at least 1.5× larger than the radial thickness (RT), such as at least 1.75× larger than the radial thickness (RT) or at least 2× larger than the radial thickness (RT).

Clause 18] The anastomosis clip (100, 200) according to clause 16 or 17,
wherein, in the closed position, a section of a right one (4) of said prongs of the first fork (2) extends parallel to the right side part (32) of the ring structure (1), whilst a section of a left one (5) of said prongs of the first fork extends parallel to the left side part (31) of the ring structure (1).

Clause 19] The anastomosis clip (100, 200) according to clause 18,
wherein, in the closed position, said section of the right prong (4) of the first fork (2) and said section of the left prong (5) of the first fork (2) lie submerged in the inner space (30) bounded by the ring structure (1).

Clause 20] The anastomosis clip (100, 200) according to one of the clauses 16-19 in combination with one of the clauses 14-15,
wherein, in the closed position, a section of a right one (54) of the prongs (54, 55) of the second fork (52) extends parallel to the right side part (32) of the ring structure (1), whilst a section of a left one (55) of the prongs (54, 55) of the second fork (52) extends parallel to the left side part (31) of the ring structure (1).

Clause 21] The anastomosis clip (100, 200) according to clause 20,
wherein, in the closed position, said section of the right prong (54) of the second fork (52) and said section of the left prong (55) of the second fork (52) lie submerged in the inner space (30) bounded by the ring structure (1).

Where the terms proximal and/or distal are used in this application, these terms are used in relation to the longitudinal direction of the prongs of the fork(s), distal corresponding to the side of the free ends of the prongs and proximal corresponding to the side of the fixed ends of the prongs.

1.-21. (canceled)

22. An anastomosis clip for connecting a first vessel to the side wall of a second vessel,
wherein the anastomosis clip comprises:
a first two prong fork having a fork base and two prongs extending from the fork base to a tapered end part, the tapered end parts tapering from a taper base to a sharp tip,
a ring structure defining a ring plane, and
a clamping structure configured to clamp the first fork, in a closed position of the first fork, with a first clamping force towards the ring structure;
wherein the ring structure and the first fork are movable with respect to each other from an open position of the first fork to the closed position of the first fork;
wherein, in the open position of the first fork, the ring structure and the first fork are arranged at a distance from each other;
wherein, in the closed position of the first fork:
the prongs of the first fork extend along the ring plane, and
the fork base of the first fork is located at a base side of the ring structure whilst the tapered end parts of the prongs of the first fork are located at a prong end side of the ring structure, the prong end side being, viewed along the ring plane, opposite the base side;
wherein the ring structure has, for each of said prongs of the first fork, an associated supporting area associated to a said prong of the first fork;
wherein each said supporting area:
has, viewed with respect to the fork base of the first fork, a distal end and a proximal end, and
is configured to support a part of the associated prong when the first fork is, in the closed position of the first fork, clamped with the first clamping force towards the ring structure;
and
wherein, in the closed position of the first fork, a first distance from the fork base of the first fork to the sharp tip of the tapered end part of a said prong is at most as long as a second distance from the fork base of the first fork to the distal end of the associated supporting area such that, viewed in a transverse direction transverse to the ring plane, each said sharp tip is shielded by its associated supporting area.

23. The anastomosis clip according to claim 22,
wherein, in the closed position of the first fork, the associated supporting area overlaps at least about 35% of the tapered end part.

24. The anastomosis clip according to claim 22,
wherein the supporting areas have, viewed from their proximal end to their distal end, an area length; and
wherein the area length is at least 2.0× the thickness of the prongs of the first fork.

25. The anastomosis clip according to claim 22,
wherein the supporting areas have, viewed from their proximal end to their distal end, an area length; and
wherein the area length is at most 6× the thickness of the prongs of the first fork.

26. The anastomosis clip according to claim 22,
wherein the supporting areas have, viewed from their proximal end to their distal end, an area length; and
wherein the area length is equal to or larger than the length of the tapered end part.

27. The anastomosis clip according to claim 22,
wherein the supporting areas have, viewed from their proximal end to their distal end, an area length; and
wherein the area length is at least 110% of the length of the tapered end part.

28. The anastomosis clip according to claim 22,
wherein, in the closed position of the first fork, a third distance from the fork base of the first fork to the taper base of the tapered end part of a said prong is at least as long as a fourth distance from the fork base of the first fork to the proximal end of the associated supporting area such that, viewed in the transverse direction, each said taper base is supported by its associated supporting area.

29. The anastomosis clip according to claim 22,
wherein the tapered end parts of the prongs of the first fork each have a first taper with a first taper direction and a second taper with a second taper direction;
wherein the first taper direction is different from the second taper direction; and
wherein, viewed in length direction of the prongs, the second taper has a shorter length than the first taper has.

30. The anastomosis clip according to claim 29,
wherein, viewed in the closed position of the first fork, the first taper direction is towards the associated supporting area and the second taper direction is away from the associated supporting area.

31. The anastomosis clip according to claim 29,
wherein the first taper direction and second taper direction are at an angle with respect to each other, such as perpendicular to each other.

32. The anastomosis clip according to claim 22,
wherein the tapered end parts of the prongs of the first fork each have a ring facing surface, which, in the closed position of the first fork, faces the associated supporting area; and
wherein, in the closed position of the first fork, the ring facing surface or at least part of the ring facing surface of each said prong of the first fork lies parallel to and optionally against the associated supporting area.

33. The anastomosis clip according to claim 22,
wherein the supporting areas are recessed in the prong end side of the ring structure.

34. The anastomosis clip according to claim 22,
wherein the clip further comprises:
a second two prong fork having a fork base and two prongs extending from the fork base to a free end part, the free end parts of the prongs of the second fork tapering to a sharp tip, and
a further clamping structure configured to clamp the second fork, in a closed position of the second fork, towards the ring structure of first fork;
wherein the ring structure and second fork are movable with respect to each other between an open position of the second fork and the closed position of the second fork;
wherein, in the open position of the second fork, the ring structure and second fork are arranged at a distance from each other; and
wherein, in the closed position of the second fork and viewed with respect to the ring plane:
the first fork is arranged at one side of the ring plane and the second fork is arranged at the other side of the ring plane,
the prongs of the second fork extend along the ring plane,
the fork base of the second fork is located at the base side of the ring structure whilst the free end parts of the prongs of the second fork are located at the prong end side of the ring structure, and
the free end parts of the prongs of the second fork lie submerged in an inner space bounded by the ring structure such that, in the closed position of the second fork, the sharp tips of the second fork are shielded by the ring structure.

35. The anastomosis clip according to claim 34,
wherein, in the closed position of the second fork, the second fork is clamped towards the first fork with a second clamping force), the second clamping force being directed opposite to the first clamping force; and
wherein the second clamping force is smaller than the first clamping force.

36. The anastomosis clip according to claim 22,
wherein the ring structure further has a left side part connecting the base side with the prong end side, and, opposite to the left side part, a right side part connecting the base side with the prong end side;
wherein the left side part and right side part have, viewed in radial direction of the ring structure, a radial thickness; and
wherein said length of the supporting areas is larger than the radial thickness.

37. The anastomosis clip according to claim 22,
wherein the ring structure further has a left side part connecting the base side with the prong end side, and, opposite to the left side part, a right side part connecting the base side with the prong end side; and
wherein, in the closed position of the first fork, a section of a right one of said prongs of the first fork extends parallel to the right side part of the ring structure, whilst a section of a left one of said prongs of the first fork extends parallel to the left side part of the ring structure.

38. The anastomosis clip according to claim 37,
wherein, in the closed position of the first fork, said section of the right prong of the first fork and said section of the left prong of the first fork lie submerged in the inner space bounded by the ring structure.

39. The anastomosis clip according to claim 35,
wherein the ring structure further has a left side part connecting the base side with the prong end side, and, opposite to the left side part, a right side part connecting the base side with the prong end side; and
wherein, in the closed position of the second fork, a section of a right one of the prongs of the second fork extends parallel to the right side part of the ring structure, whilst a section of a left one of the prongs of the second fork extends parallel to the left side part of the ring structure.

40. The anastomosis clip according to claim 39,
wherein, in the closed position of the second fork, the second fork is clamped towards the first fork with a second clamping force), the second clamping force being directed opposite to the first clamping force; and
wherein the second clamping force is smaller than the first clamping force.

41. The anastomosis clip according to claim 39,
wherein, in the closed position of the second fork, said section of the right prong of the second fork and said section of the left prong of the second fork lie submerged in the inner space bounded by the ring structure.
